SEQUENCE LISTING (28 sequences)

SEQ ID NO: 1 - RNA, 22 nt, Artificial Sequence
  source: /note=“Description of Artificial Sequence Synthetic oligonucleotide”
auguucaucacaguaauaggag

SEQ ID NO: 2 - RNA, 22 nt, Artificial Sequence
  source: /note=“Description of Artificial Sequence Synthetic oligonucleotide”
aguauggucuacgcauauucuc

SEQ ID NO: 3 - protein, 13 aa, Artificial Sequence
  source: /note=“Description of Artificial Sequence Synthetic peptide”
AKFVAAWTLKAAA

SEQ ID NO: 4 - DNA, 5919 bp, Homo sapiens
atgggggcctcttctccaagaagcccggagcctgtcgggccgccggcgcccggtctccccttctgctgcggaggatccctgctggcggttgtggtgctgcttgcgctgccggtggcctggggtcaatgcaatgccccagaatggcttccatttgccaggcctaccaacctaactgatgaatttgagtttcccattgggacatatctgaactatgaatgccgccctggttattccggaagaccgttttctatcatctgcctaaaaaactcagtctggactggtgctaaggacaggtgcagacgtaaatcatgtcgtaatcctccagatcctgtgaatggcatggtgcatgtgatcaaaggcatccagttcggatcccaaattaaatattcttgtactaaaggataccgactcattggttcctcgtctgccacatgcatcatctcaggtgatactgtcatttgggataatgaaacacctatttgtgacagaattccttgtgggctaccccccaccatcaccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacctgggcaggaagtgttctacagctgtgagcccggctacgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttactgtgtcttggctggaatggaaagcctttggaatagcagtgttccagtgtgtgaacaaatcttttgtccaagtcctccagttattcctaatgggagacacacaggaaaacctctggaagtctttccctttgggaaaacagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcgggcaatgctgcccattggagcacgaagccgccaatttgtcaacgaattccttgtgggctaccccccaccatcgccaatggagatttcattagcaccaacagagagaattttcactatggatcagtggtgacctaccgctgcaatcctggaagcggagggagaaaggtgtttgagcttgtgggtgagccctccatatactgcaccagcaatgacgatcaagtgggcatctggagcggcccggcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccggagctaccaagctgctccagggtatgtcagccacctccagatgtcctgcatgctgagcgtacccaaagggacaaggacaacttttcacccgggcaggaagtgttctacagctgtgagcccggctatgacctcagaggggctgcgtctatgcgctgcacaccccagggagactggagccctgcagcccccacatgtgaagtgaaatcctgtgatgacttcatgggccaacttcttaatggccgtgtgctatttccagtaaatctccagcttggagcaaaagtggattttgtttgtgatgaaggatttcaattaaaaggcagctctgctagttattgtgtcttggctggaatggaaagcctttggaatagcagtgttccagtgtgtgaacaaatcttttgtccaagtcctccagttattcctaatgggagacacacaggaaaacctctggaagtctttccctttggaaaagcagtaaattacacatgcgacccccacccagacagagggacgagcttcgacctcattggagagagcaccatccgctgcacaagtgaccctcaagggaatggggtttggagcagccctgcccctcgctgtggaattctgggtcactgtcaagccccagatcattttctgtttgccaagttgaaaacccaaaccaatgcatctgactttcccattgggacatctttaaagtacgaatgccgtcctgagtactacgggaggccattctctatcacatgtctagataacctggtctggtcaagtcccaaagatgtctgtaaacgtaaatcatgtaaaactcctccagatccagtgaatggcatggtgcatgtgatcacagacatccaggttggatccagaatcaactattcttgtactacagggcaccgactcattggtcactcatctgctgaatgtatcctctcaggcaatactgcccattggagcacgaagccgccaatttgtcaacgaattccttgtgggctacccccaaccatcgccaatggagatttcattagcaccaacagagagaattttcactatggatcagtggtgacctaccgctgcaatcttggaagcagagggagaaaggtgtttgagcttgtgggtgagccctccatatactgcaccagcaatgacgatcaagtgggcatctggagcggccccgcccctcagtgcattatacctaacaaatgcacgcctccaaatgtggaaaatggaatattggtatctgacaacagaagcttattttccttaaatgaagttgtggagtttaggtgtcagcctggctttgtcatgaaaggaccccgccgtgtgaagtgccaggccctgaacaaatgggagccagagttaccaagctgctccagggtgtgtcagccgcctccagaaatcctgcatggtgagcataccccaagccatcaggacaacttttcacctgggcaggaagtgttctacagctgtgagcctggctatgacctcagaggggctgcgtctctgcactgcacaccccagggagactggagccctgaagccccgagatgtgcagtgaaatcctgtgatgacttcttgggtcaactccctcatggccgtgtgctatttccacttaatctccagcttggggcaaaggtgtcctttgtctgtgatgaagggtttcgcttaaagggcagttccgttagtcattgtgtcttggttggaatgagaagcctttggaataacagtgttcctgtgtgtgaacatatcttttgtccaaatcctccagctatccttaatgggagacacacaggaactccctctggagatattccctatggaaaagaaatatcttacacatgtgacccccacccagacagagggatgaccttcaacctcattggggagagcaccatccgctgcacaagtgaccctcatgggaatggggtttggagcagccctgcccctcgctgtgaactttctgttcgtgctggtcactgtaaaaccccagagcagtttccatttgccagtcctacgatcccaattaatgactttgagtttccagtcgggacatctttgaattatgaatgccgtcctgggtattttgggaaaatgttctctatctcctgcctagaaaacttggtctggtcaagtgttgaagacaactgtagacgaaaatcatgtggacctccaccagaacccttcaatggaatggtgcatataaacacagatacacagtttggatcaacagttaattattcttgtaatgaagggtttcgactcattggttccccatctactacttgtctcgtctcaggcaataatgtcacatgggataagaaggcacctatttgtgagatcatatcttgtgagccacctccaaccatatccaatggagacttctacagcaacaatagaacatcttttcacaatggaacggtggtaacttaccagtgccacactggaccagatggagaacagctgtttgagcttgtgggagaacggtcaatatattgcaccagcaaagatgatcaagttggtgtttggagcagccctccccctcggtgtatttctactaataaatgcacagctccagaagttgaaaatgcaattagagtaccaggaaacaggagtttctttaccctcactgagatcatcagatttagatgtcagcccgggtttgtcatggtagggtcccacactgtgcagtgccagaccaatggcagatgggggcccaagctgccacactgctccagggtgtgtcagccgcctccagaaatcctgcatggtgagcataccctaagccatcaggacaacttttcacctgggcaggaagtgttctacagctgtgagcccagctatgacctcagaggggctgcgtctctgcactgcacgccccagggagactggagccctgaagcccctagatgtacagtgaaatcctgtgatgacttcctgggccaactccctcatggccgtgtgctacttccacttaatctccagcttggggcaaaggtgtcctttgtttgcgatgaagggttccgattaaaaggcaggtctgctagtcattgtgtcttggctggaatgaaagccctttggaatagcagtgttccagtgtgtgaacaaatcttttgtccaaatcctccagctatccttaatgggagacacacaggaactccctttggagatattccctatggaaaagaaatatcttacgcatgcgacacccacccagacagagggatgaccttcaacctcattggggagagctccatccgctgcacaagtgaccctcaagggaatggggtttggagcagccctgcccctcgctgtgaactttctgttcctgctgcctgcccacatccacccaagatccaaaacgggcattacattggaggacacgtatctctatatcttcctgggatgacaatcagctacatttgtgaccccggctacctgttagtgggaaagggcttcattttctgtacagaccagggaatctggagccaattggatcattattgcaaagaagtaaattgtagcttcccactgtttatgaatggaatctcgaaggagttagaaatgaaaaaagtatatcactatggagattatgtgactttgaagtgtgaagatgggtatactctggaaggcagtccctggagccagtgccaggcggatgacagatgggaccctcctctggccaaatgtacctctcgtacacatgatgtctaa

SEQ ID NO: 5 - protein, 302 aa, Homo sapiens
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 6 - protein, 25 aa, Homo sapiens
EDPGKQLYNVEATSYALLALLQLKD

SEQ ID NO: 7 - protein, 25 aa, Artificial Sequence
  source: /note=“Description of Artificial Sequence Synthetic peptide”
EDPGKQLYNVEATSYALLALLQLKD

SEQ ID NO: 8 - protein, 29 aa, Homo sapiens
GSTQATFMVFQALAQYQKDAPDHQELNLD

SEQ ID NO: 9 - protein, 29 aa, Artificial Sequence
  source: /note=“Description of Artificial Sequence Synthetic peptide”
GSTQALFMVFQAIAQYQKDAPDHQELNLD

SEQ ID NO: 10 - protein, 26 aa, Homo sapiens
KRAPSTWLTAYVVKVFSLAVNLIAID

SEQ ID NO: 11 - protein, 26 aa, Artificial Sequence
  source: /note=“Description of Artificial Sequence Synthetic peptide”
KRAPSTILTAYVVKVFSLAVNLIAID

SEQ ID NO: 12 - protein, 30 aa, Homo sapiens
EKDMALTAFVLISLQEAKDICEEQVNSLPG

SEQ ID NO: 13 - protein, 30 aa, Artificial Sequence
  source: /note=“Description of Artificial Sequence Synthetic polypeptide”
EKDMALTAFVIISLQEAKDICEEQVNSLPG

SEQ ID NO: 14 - protein, 28 aa, Homo sapiens
KVFSLAVNLIAIDSQVLCGAVKWLILEK

SEQ ID NO: 15 - protein, 28 aa, Artificial Sequence
  source: /note=“Description of Artificial Sequence Synthetic peptide”
KVFSLAVNLIAIDSQVLCGAVKWLILEK

SEQ ID NO: 16 - DNA, 30 bp, Artificial Sequence
  source: /note=“Description of Artificial Sequence Synthetic oligonucleotide”
cgatgacatatggaacggacaacgaggact

SEQ ID NO: 17 - DNA, 30 bp, Artificial Sequence
  source: /note=“Description of Artificial Sequence Synthetic oligonucleotide”
ggacactgtgcagtctctaacccagggaga

SEQ ID NO: 18 - DNA, 30 bp, Artificial Sequence
  source: /note=“Description of Artificial Sequence Synthetic oligonucleotide”
cggcgctggcacgagaacctcatggggttc

SEQ ID NO: 19 - RNA, 19 nt, Artificial Sequence
  source: /note=“Description of Artificial Sequence Synthetic oligonucleotide”
acauucugauuccuuccgg

SEQ ID NO: 20 - RNA, 19 nt, Artificial Sequence
  source: /note=“Description of Artificial Sequence Synthetic oligonucleotide”
acuuucugcacuccuucac

SEQ ID NO: 21 - DNA, 104 bp, Artificial Sequence
  source: /note=“Description of Artificial Sequence Synthetic polynucleotide”
acattctgattccttccggaagccggaaggaatcagaatgacattttttgagctcaaaaaatgtctttctgcactccttcaccttgtgaaggagtgcagaaagt

SEQ ID NO: 22 - DNA, 1119 bp, Artificial Sequence
  source: /note=“Description of Artificial Sequence Synthetic polynucleotide”
atgactgtagctagaccctctgtgcctgcagctctgcccttgctcggagagctgcccaggctcctcctcctggtactcctttgtttgccggcggtgtggggactcgacgcggagcgactgaagcatttgatcgtcacgccctctggctgcggagaacaaaatatgatcggcatgactccaaccgttatagcagttcactacttggacgagacagaacagtgggagaagtttggacttgaaaagcggcaaggagctctggagttgattaagaaaggctatacccagcaactcgcctttaggcaaccttcctcagcgttcgcagcattcgtcaagcgagccccatccacttggctcaccgcttatgttgtgaaggtcttcagcctggccgtcaacttgatagcaatcgattcccaggttctgtgtggtgccgtgaaatggcttatccttgagaagcaaaaacccgatggagtcttccaggaggacgctccagtgattcatcaggaaatgattggagggcttagaaataataacgaaaaagacatggccctgactgccttcgttttgatatcattgcaggaagccaaagacatctgtgaagaacaggtcaattcactgccaggcagtatcacgaaagctggagattttctggaggccaactacatgaacttgcaacgaagctatactgtcgcaattgcaggatatgctctggcacaaatggggcgattgaagggcccgctgcttaataagttccttaccacggctaaggataaaaatcgatgggaagaccctgggaagcagctgtacaatgtagaagcgacttcttacgcactgttggcgttgctccaattgaaagattttgattttgttccgccagttgttcggtggttgaacgaacaacggtactacggaggaggttacggatccacgcaagcaacgtttatggttttccaagcacttgcacaataccaaaaagacgcgcccgaccatcaagagctcaatctcgacgtaagcctgcagttgagcggcacgaccagcgggacgactcgcctgctttcaggtcacacgtgctttactctgacgggcctcctcggaacactcgtcactatgggtctgttgaca

SEQ ID NO: 23 - protein, 34 aa, Homo sapiens
MTVARPSVPAALPLLGELPRLLLLVLLCLPAVWG

SEQ ID NO: 24 - protein, 306 aa, Homo sapiens
LDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQL

SEQ ID NO: 25 - protein, 33 aa, Homo sapiens
SGTTSGTTRLLSGHTCFTLTGLLGTLVTMGLLT

SEQ ID NO: 26 - protein, 25 aa, Homo sapiens
MGIQGGSVLFGLLLVLAVFCHSGHS

SEQ ID NO: 27 - protein, 28 aa, Homo sapiens
ENGGTSLSEKTVLLLVTPFLAAAWSLHP

SEQ ID NO: 28 - protein, 7 aa, Artificial Sequence
  source: /note=“Description of Artificial Sequence Synthetic peptide”
SRDGRRS

(19) United States
(12) Patent Application Publication
(10) Pub. No.: US 2020/0262879 A1
(43) Pub. Date: Aug. 20, 2020

(54) METHODS AND COMPOSITIONS TO ENHANCE THE IMMUNOGENICITY OF TUMORS

(71) Applicant: INSIDEOUTBIO, INC., Charlestown, MA (US)

(72) Inventor: Alan Gordon Herbert, Charlestown, MA (US)

(21) Appl. No.: 16/645,646

(22) PCT Filed: Sep. 11, 2018
(86) PCT No.: PCT/US2018/050330

§ 371 Date: Mar. 9, 2020

Related U.S. Application Data
(60) Provisional application No. 62/656,495, filed on Apr. 12, 2018.
(60) Provisional application No. 62/582,365, filed on Nov. 7, 2017.
(60) Provisional application No. 62/556,836, filed on Sep. 11, 2017.

(51) Int. Cl. C07K 14/47; A61K 31/7105; C07K 14/705

(52) U.S. Cl. CPC C07K 14/472; A61K 9/51; C07K 14/70596; A61K 31/7105

(57) ABSTRACT

Methods and compositions for enhancing the immunogenicity of a tumor of interest by modulating/altering the expression of specific complement proteins and complement protein receptors associated with the immune suppression of a tumor/tumor cell are described herein.

### RELATED APPLICATIONS

This application claims the benefit under 35 U.S.C. § 119(e) of U.S. Provisional Application Nos. 62/556,836, filed on Sep. 11, 2017; 62/582,365, filed on Nov. 7, 2017; and 62/656,495, filed on Apr. 12, 2018, all of which are incorporated herein by reference in their entirety.

### INCORPORATION BY REFERENCE OF MATERIAL IN ASCII TEXT FILE

The instant application contains a Sequence Listing which has been submitted electronically in ASCII format and is hereby incorporated by reference in its entirety. Said ASCII copy, created on Sep. 10, 2018, is named 0371_0001WO1_SL.txt and is 22,560 bytes in size.

### BACKGROUND OF THE INVENTION

Cancerous tumors create a shield of invisibility to protect themselves from the body's immune-driven defense mechanisms. Various methods have been developed to enhance the immune response to such tumors, including inoculation with bacterial infections; viruses; vaccines prepared from tumor cells; immune-stimulants or adjuvants; immune-modulators that boost the immune response and inhibitors of metabolic enzymes that suppress the immune response. The rationale for these approaches is that once the tumor is no longer hidden and the immune system is engaged, tumor cells will be susceptible to elimination by the body's immune response like other pathogens. Despite progress, however, there remains a need for immunomodulatory methods that specifically target tumor cells and enhance tumor immunogenicity as a means to effectively inhibit tumor growth and metastasis and to provide new options to treat or prevent cancer.

### SUMMARY OF THE INVENTION

The present invention encompasses methods and compositions for the modulation of complement protein production and/or expression in a tumor cell to inhibit complement-driven tumor cell growth and metastasis. The invention further comprises methods to activate, or enhance, the immune response against tumor cells. As described herein, the methods of the present invention take all or part of the tumor out of stealth mode, switching the treated tumor from “cold” mode, with limited immune response and low levels of cytolytic enzymes like granzyme B and perforin 1, to “hot” mode where the tumor induces a response that makes all parts of the tumor susceptible to attack by the body's immune system.

Complement proteins, complement protein receptors and proteolytic enzymes associated with the complement activation pathway have been investigated for their roles in cancer and in tumor growth and metastasis. In particular, complement proteins C3, C5 (and their degradation/proteolytic fragments such as iC3b), and complement cell surface receptors C3aR and C5aR are molecules associated with the immunosuppression of a tumor. Complement protein C3d or C3dg, are breakdown products of the C3 protein and have been associated with the immunostimulatory activity of a tumor, i.e., making the tumor more susceptible to an immune response. Thus, as provided for in this invention, decreasing the expression of, or activity of, complement proteins C3, C3a, C5 and C5a to prevent production of immunosuppressive break down degradation products such as iC3b, while increasing the expression of (or over-expression of), production of; or increasing the activity of immunostimulatory degradation products such as C3d or C3dg or other immunostimulatory peptides on the tumor cell surface or in the local tumor micro-environment provides a basis for greatly enhancing the immunogenicity of the tumor.

Production of C3 immunosuppresive degradation products near or on the tumor surface can also be amplified using pathway components synthesized by the tumor or from components, such as CFH, that capture C3 from the microenvironment and recycle it to the tumor surface processing within the tumor (Martin, Leffler et al. 2016; Elvington, Liszewski et al. 2017). C3 degradation may occur through proteases external to the cell or use proteases internal such as those belonging to the cathepsin family (Liszewski, Kolev et al. 2013; Martin, Leffler et al. 2016; Elvington, Liszewski et al. 2017). In either case, C3b secreted by the tumor cell can be further amplified by pathways external to the tumor to increase deposition of inhibitory C3 degradation products on the tumor surface. Binding of CFH to C3b also acts to inhibit production of immunostimulatory products such as C3d by blocking the access to the proteolytic sites on C3b necessary for production of C3d (Xue et al. 2017, FIG. 1).

Methods of enhancing the immunogenicity of a tumor of interest, or of cancer or tumor cells, by modulating production or altering the expression of specific complement proteins and/or complement factors and/or complement protein receptor(s) and/or complement relevant proteolytic enzymes associated with immunosuppression of a tumor cell are described herein. Specific targets as described herein can be complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CF1 and CFP; complement regulators such as C1QBP, CD46, CD55 and CD59; or cathepsins such as. CTSB, CTSC, CTSD, CSTL, CSTO or CTSS or any combination thereof.

In particular, the tumors of interest comprise tumor cells that express complement components such as complement protein C3 or C5. Alternatively, the tumor cells can also express complement receptors such as complement protein receptor C3aR1 or C5aR1. Other complement receptors or complement-associated receptors that can be modulated for the purposes of this invention include, for example, C5aR2, C1R, C1RL, CR2, C1QBP, CD46, CD55, CD59, and LAIR1, and also, for example, complement factors such as CFB, CFD, CFH, CFHR1, CFHR2, CFHR3, CFHR4, CFHR5, CFI and CFP. Some proteolytic enzymes produced by tumor cells, such as cathepsins (such as CTSB, CTSC, CTSD, CSTL, CSTO, or CBS or any combination thereof) can also be modulated. See for example, Chauhan, S. et al. Cancer Res. 51. 1478-1481 (Mar. 1, 1991).

The tumor or cancer cells of interest can express one, or any combination of the above-mentioned complement proteins, complement receptors, complement factors, complement regulators or cathepsins. Thus, complement components C3, C5, or any of their respective proteolytic degradation products, or their respective cell surface receptors, are all suitable for the methods described herein.

As described herein, one aspect of enhancing the immunogenicity of a tumor of interest involves using a combination of agents to be administered as a therapeutic to decrease, or inhibit (partially or completely abrogate) the expression or activity of complement proteins C3, C5, or expression or signaling activity of complement receptors or complement regulatory proteins such as complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof that lead to immunosuppressive breakdown products of C3, while also increasing the expression/production or activity of the immunostimulatory complement degradation products C3d or C3dg or other immunostimulatory peptides either by the tumor cells or other cells in the tumor microenvironment. Such modulation of protein production, activity or expression can be performed substantially concurrently or sequentially.

Methods described herein include the steps of contacting a tumor cell with a first agent, wherein the first agent decreases the expression of, or production of, for example, complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHRS, CFI and CFP; complement regulators such as C1QBP, CD46, CD55 and CD59; or cathepsins such as CTSB, CTSC, CTSD, CSTL, CSTO, or CTSS or any combination thereof, in the tumor cell and also contacting the tumor cell with a second agent wherein the second agent increases the expression, activity or production of complement protein C3d in the tumor cells or the expression, activity or production of other immunostimualtory peptides. Contacting the tumor cell with the first agent can occur prior to, substantially concurrently with, or after contacting the tumor cell with the second agent.

In one embodiment, the first agent comprises a gene-editing agent that decreases or inhibits the expression of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof within the tumor cells. The gene-editing agent can comprise a CRISPR (Clustered Regularly Interspaced Short Palindromic Repeats) system construct that decreases or inhibits the expression of one, or more, complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell. Alternatively, the gene-editing agent can comprise a TALEN (Transcription Activator-like Effector Nucleases) construct that decreases or inhibits the expression of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell, a meganuclease, homologous recombination or base editing. As described herein, the gene-editing agent can be constructed so that expression of complement components, such as C3 and C5; complement receptors such as C3aRL C5aR1, C5alt2C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof is decreased or inhibited, but the expression, activity or production of C3d or other immunostimulatory peptides in the tumor cell is not.

In another embodiment of the present invention, the first agent is a nucleic acid construct comprising RNAi, shRNA, mi RNA or anti-sense RNA that decreases or inhibits the expression of one, or more complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereofin the tumor cell. In yet another embodiment, the first agent is a nucleic acid construct that expresses a protein that decreases or inhibits the transcription of one, or more complement components, such as C3 and C5; complement receptors such as C3aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereotin the tumor cell. The agent may be targeted for delivery to the tumor cell using known delivery vehicles, including without limitation, viral vectors, nanoparticles, liposomes or exosomes. If the delivery of the construct is via a viral vector, the viral vector can comprise any suitable replicating or non-replicating viral vector for targeting and delivery of the construct into a tumor cell and can be for example, adenovirus, adeno-associated virus, a lentiviral vector, a vaccinia virus, a herpes virus vector, a paromxyovirusor or any viral vector or any virus-like particle.

Another embodiment of the present invention is the use of inhibitors of the C3 convertase complex generated by either the classical, alternative or lectin pathway or by other proteolytic enzymes capable of generating this complex. Inhibition of C3 convertase complex inhibits the enzymatic breakdown of C3 into C3a and C3b. The convertase inhibitors can comprise for example, soluble complement receptor 1, referred to herein as sCR1 (“Soluble human complement receptor type 1: in vivo inhibitor of complement suppressing post-ischemic myocardial inflammation and necrosis” Weisman et al. Science 1990 Jul. 13; 249 (4965):146-51); See FIG. 6 for the nucleic acid sequence of sCR1). Another C3 convertase conversion complex inhibitor can comprise Complementation Activation Blocker-2 (CAB-2; “A soluble chimeric complement inhibitory protein that possesses both decay-accelerating and factor I cofactor activities” Higgins et al., J Immunol, 1997 Mar. 15; 158 (6):2872-81 and “Modulation and repletion/enhancement of the complement system for treatment of trauma” US 2011019022.1 A1). Additional C3 convertase complex inhibitors can comprise fusion proteins made from combinations of known complement receptors. See, for example, “Design and development of TT30, a novel C3d-targeted C3/C5 convertase inhibitor for treatment of human complement alternative pathway-mediated diseases”, Fridkis-Harel et al, Blood, 27 Oct. 2011, vol. 118, number 17; “Regional Engineering of a Minimized Inhibitor with Unique Triple-Targeting Properties”, Schmidt, et al., J. immonol., 2013, 190: 5712-5721 (describing a complelment regulator factor H “mini-FH” construct) and “Polypeptides for inhibiting complement Activation” WO 2017/109208. The approach specifically involves encoding these inhibitors as nucleic acids that are then expressed in the tumor microenvironment using techniques known to those experienced in the art, such as recombinant adenovirus, adeno-associated virus, a lentiviral vector, a vaccinia virus, a herpes virus vector, a paromxyovirusor or any viral vector or any virus-like particle or by the use of plasmids or min-circles. The vectors are designed so as not to affect the expression, production or activity of C3d or other immunostimulatory peptides in the tumor cell or its micro-environment but to diminish the production of immunosuppressive breakdown products of C3.

Alternatively, as described herein, the complement factors (CFB, CFD, CFH, CFHR1, CFHR2, CFHR3, CFHR4, CFI, CFP) are also suitable targets for the therapies described herein. wherein decreasing the expression of one, or more, these factors, or inhibiting their activity, results in enhancing the immunogenicity of tumors by increasing production of immunostimulatory products susch as C3d (FIG. 1). Production of both C3 and CFH, are dysregulated (upregulated) in models of aggressive cancer (see e.g., JnBaptiste Gurtan et al. (2017) wherein Supplemental Table S6 illustrates that these genes are part of a signature that predicts poor survival). Sequences that bind C3d are conserved between complement factors e.g., CFH and CFHR3. CFHR3 can inhibit stimulation of B-Cells (Fritsche, Lauer et al. 2010). The conservation between CFH and CFHR3 is at the nucleotide level, allowing RNA interference in the translation of both transcripts with a single gRNA. Examples of such RNAi sequences are AUGUUCAUCACAGUAAUAGGAG (SEQ ID NO: 1) and AGUAUGGUCUACGCAUAUUCUC (SEQ ID NO: 2). Genes for both these components could also be simultaneously knocked-out using a gene editing approach using guides targeted at these conserved sequences. Loss of both these proteins will increase the immunogenecity of tumor cells as the production and activity of Cad will no longer be inhibited by the tumor.

The methods of the present invention further comprise contacting mor or cancer cell with a second agent, either prior to, concurrently, substantially simultaneously, or after contact with the first agent. The second agent increases, initiates or stimulates the production or expression of a complement component or other immunostimulatory peptides that are associated with the immunostimulation or increased immunosurviellance of the tumor. In particular, the second agent comprises an expression vector that targets the tumor cell, wherein the vector comprises a nucleic acid construct that expresses C3d or peptides derived from C3d, or a biologically active variant thereof, or encodes a protein that activates expression of C3d, in the tumor cell including mutants that increase binding to Class I or Class II Major Histocompatibility (MHC) antigens and that remove the cysteine at the thio-ester site in the C3d domain. As a result of contacting a tumor cell with the combination of the two agents, the immunogenicity of the tumor cell is enhanced and the tumor cell becomes more susceptible to attack by the immune system.

In another embodiment of the present invention, the second agent can comprise an immunostimulatory protein or peptide that can bind to multiple Class I and/or Class II NI IC alleles to stimulate an immune response against a broad range of tumors in individuals with different genetic backgrounds. An example of such a peptide is PADRE, or pan HLA-DR epitope peptide. (See e.g., Alexander, J. et al. Immunity, vol. 1, 751-761, December 1994; Song, L. et al. PloS ONE 9 (12) 2014.) In particular, the pan-stimulatory peptide PADRE (AKFVAAWTLKAAA (SEQ ID NO: 3) can be used as an alternative to C3d as a second agent in the methods described herein, in conjunction with the first agent that knocks-down or knocks-out C3 and/or other complement or complement associated proteins as described herein.

Also encompassed by the present invention are methods of inhibiting tumor growth in a subject, wherein the tumor comprises cells that express complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof said proteins or receptors thereof, the method comprising administering to the subject a therapeutically effective amount of a first agent wherein the first agent decreases the expression of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, in the tumor cells, and administering to the subject a therapeutically effective amount of a second agent wherein the second agent increases the expression of complement protein C3d or C3d derived peptides, or other immunostimulatory peptides in the tumor cells or in the tumor micro-environment, thereby inhibiting the tumor growth in the subject. Administration of the first agent may occur prior to, substantially simultaneously or subsequently to administration of the second agent.

In a particular embodiment, the subject in the methods of this invention is a mammal, and more particularly, the mammal is a human. The first and second agents of this method are as described above.

A particular embodiment of the present invention encompasses methods of treating cancer, or preventing metastasis of cancer, in a subject (in the case of a human subject, also referred to herein as an individual or patient), wherein the tumor cells of the cancer express complement protein complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof said proteins or receptors thereof, the method comprising administering to the individual a therapeutically effective amount of a first agent wherein the first agent decreases the expression of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, in the cancer cells, and administering to the individual a therapeutically effective amount of a second agent wherein the second agent increases the expression, activity or production of complement protein C3d or C3d derived peptides, or other immunostimulatory peptides, in the tumor cells or in the tumor micro-environment, thereby treating cancer, or preventing metastasis of cancer, in the subject. Administration of the first agent may occur prior to, substantially simultaneously or subsequently to administration of the second agent. The first and second agents of this method are as described above.

Any cancer in a subject can be treated by the methods described herein as long as the tumor cells of the cancer are associated with the autocrine complement pathway (FIG. 5A-C), and in particular, express one or more of the complement components as described herein. For example, the cancer can be ovarian, breast, renal, prostate, lung, colon or lung cancer.

The method of treating cancer can further encompass administering the first and/or second agent concurrently with, or sequentially before or after, or in conjunction with, at least one, or more additional or complementary cancer treatments suitable for the treatment of the specific cancer. For example, without limitation, the complementary cancer treatment can be selected from a therapy comprising checkpoint inhibitor; a proteasome inhibitor; immunotherapeutic agent; radiation therapy or chemotherapy. Other suitable additional or complementary cancer therapies are known to those of skill in the art.

Also encompassed by the present invention is a pharmaceutical composition, or compositions, comprising a therapeutically effective amount of a first agent and a therapeutically effective amount of a second agent as described herein. Although the composition can comprise both the first and second agent, an alternative embodiment encompasses two compositions (one comprising the first agent and one comprising the second agent) that can be administered substantially simultaneously or sequentially. In both pharmaceutical composition embodiments, the first agent decreases the expression of complement components, such as C3 and C5; complement receptors such as C3aR1, C5aR1, C5aR2, C1R, C1RL, CR2 and LAIR1; complement factors such as CFI3, CFI), CFH, CFHRT, CFHR2, CFHR3, CFHR11, CFHR5, CFI and CFP; complement regulators such as C1QBP, CD46, CD55 and CD59; or cathepsins such as CTSB, CTSC, CTSD, CSTL, CSTO, or CTSS or any combination thereof, in tumor cells, the second agent increases the expression of complement protein C3d or C3d derived, or other immunostimulatory, biologically active peptides in the tumor cells or in the tumor micro-environment. The composition additionally can include a pharmaceutically acceptable medium, suitable as a carrier for the first and second agent. The compositions can also include targeting agents to deliver the compositions to specific tumor sites.

The above and other features of the invention including various novel details of construction and combinations of parts, and other advantages, will now be more particularly described with reference to the accompanying drawings and pointed out in the claims. It will be understood that the particular methods and compositions embodying the invention are shown in the drawings and examples by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or application publication with color drawings will be provided by the Office upon request and payment of the necessary tee. Of the drawings:

FIG. 1 is a schematic that depicts the regulation of iC3b and C3dg generation by binding site of cofactors. The upper branch of the pathway leads to immunosuppressive products such as iC3b while the lower branch produces itnmunostitnulatory products such as C3d. In this depiction, CFH prevents access to a proteolytic site that leads to C3dg formation, favoring deposition of immunosuppressive iC3b while CR1 leaves this site exposed, favoring formation of C3d and immunostimulation (Nature Structural & Molecular Biology 24, 643-651 (2017).

FIG. 2A depicts constructs suitable for use in the described methods. Adeno associated virus (AAV) constructs are used to deliver CRISPR to cells to knock-out the C3 genes. sgRNAs do not include the C3d sequence (vector 1). Multiple copies of C3d, or engineered variants or other immunostimulatory peptides, are delivered to the same cell to direct production of these peptides (vector 2). The AAV vectors are pseudotyped with capsid proteins controlling delivery to the tumor cells.

FIG. 2B depicts constructs suitable for use in the described methods. Adeno associated virus (AAV) constructs are used to deliver AGO nuclease to cells to knock-out the C3 genes (Construct 1). gRNAs do not include the C3d sequence (Construct 2). C3d, or engineered variants or other immunostimulatory peptides, are delivered to the membrane of the same cell to direct production of these peptides (Construct 3). Although shown seprately, the constructs in some cases can be combined and used in viruses able to accommodate large sized inserts.

FIG. 3 depicts structural transitions of complement component C3 and its activation products. Nishida N1, Walz T, Springer T A. Proc Natl Acad Sci USA. 2006 Dec. 26; 103 (52):19737-42. C3b is proteolytically clipped to produce many different fragments, including C3d. The site of the thioester bond is depicted by a colored circle.

FIG. 4A-B depicts the nucleic acid sequence of Homo sapiens complement C3b/C4b receptor 1 (sCR1) Transcript variant F, mRNA-extracellular domain (SEQ ID NO:4). NCBI Reference Sequence: NM-000573.3 (Weisman et. al. Science, 1990 July 13:249 (4965):145-51).

FIG. 5A-C depicts examples of complement components produced by a tumor cell (A) or taken up from the micro-environment (B). Both pathways can be amplified using components external to the tumor (C). Plus signs indicate positive feedback autocrine loops while minus signs show negative feedback.

FIG. 6 depicts C3 wild type amino acid sequences (SEQ ID NOS: 6, 8, 10, 12 and 14) and their related mutated sequences (SEQ ID NOS: 7, 9, 11, 13 and 15).

FIG. 7 depicts exemplary guide RNA sequences for CRISPR (SEQ ID NOS: 16, 17 and 18).

FIG. 8 depicts the nucleic acid sequence of a Cd3/CD55 construct encoding fusion protein (SEQ ID NO: 22).

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The publications discussed throughout the text are provided solely for their disclosure prior to the filing date of the present application. Nothing herein is to be construed as an admission that the inventors are not entitled to antedate such disclosure by virtue of prior disclosure.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated.

The terms “vector”, “vector construct” and “expression vector” mean the vehicle by which a DNA or RNA sequence (e.g. a foreign gene) can be introduced into a host cell, so as to transform the host and promote expression (e.g, transcription and translation) of the introduced sequence, Vectors typically comprise the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

In one embodiment, the viral vector can be a replication competent retroviral vector capable of infecting only replicating tumor cells with particular mutations. In one embodiment, a replication competent retroviral vector comprises an internal ribosomal entry site (IRES) 5′ to the heterologous polynucleotide encoding, e.g., a cytosine deaminase, miRNA, siRNA, cytokine, receptor, antibody or the like. When the heterologous polynucleotide encodes a non-translated RNA such as siRNA, miRNA or RNAi then no IRES is necessary, but may be included for another translated gene, and any kind of retrovirus (see below) can be used. In one embodiment, the polynucleotide is 3′ to an ENV polynucleotide of a retroviral vector. In one embodiment the viral vector is a retroviral vector capable of infecting targeted tumor cells multiple times (5 or more per diploid cell).

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

The terms “gene editing” or “gene editing techniques” as described herein can include RNA-mediated interference (referred to herein as RNAi, or interfering RNA molecules), or Short Hairpin RNA (shRNA) or CRISPR-Cas9 and TALEN. See e.g., Agrawal. N. et al., Microbiol Mol Biol Rev. 2003 December; 67 (4): 657-685; Moore, C. B., et al, Methods Mol Biol. 2010; 629: 141-158; Doudna, J. A, and Charpentier, E. Science vo. 346, 28 Nov. 2014; Sander, J. D. and Joung, K. Nature Biotech 32, 347-355 (2014); U.S. Pat. No. 8,697,359; Nemudryo, A. A. ACTA Naturae vol. 6, No. 3 (22) 2014, Anti-sense RNA can also be used. (Gleave, M. and Monia, B., Nature Reviews Cancer 5, 468-479 (June 2005)). The term “gene therapy” generally means a method of therapy wherein a desired gene/genetic sequence is inserted into a cell or tissue (along with other sequences necessary for the expression of the specific gene). See, for example, genetherapynet.com for description of gene therapy techniques.

The term “subject” as used herein can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a. “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “cancer” or “tumor” includes, but is not limited to, solid tumors and blood borne tumors. These terms include diseases of the skin, tissues, organs, bone, cartilage, blood and vessels. These terms further encompasses primary and metastatic cancers. Biomarkers identifying the expression of complement components, such as C3 and C5; complement receptors such as C3aR1, C5aR1, C5aR2, C1R, C1RL, CR2 and LAIR1; complement factors such as CFB, CFD, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in tumors provide one means of selecting patients for treatment, whether the biomarker is detected by RNA expression, antibody or other reagents that allow quantitation of these molecules,

The methods and compositions of the present invention may be used to treat any type cancerous tumor or cancer cells. Such tumors/cancers may be located anywhere in the body, including without limitation in a tissue selected from brain, colon, urogenital, lung, renal, prostate, pancreas, liver, esophagus, stomach, hematopoietic, breast, thymus, testis, ovarian, skin, bone marrow and/or uterine tissue. Cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidennoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

A “therapeutically effective” amount as used herein refers to an amount sufficient to have the desired biological effect (for example, an amount sufficient to decrease the expression of complement components, such as C3 and C5; complement receptors such as C3aR1, C5aR1, C5aR2, C1R, C1RL, CR2 and LAIR1; complement factors such as CFB, CFD, CFH, CFHR2, CFHR3, CFHR4, CFHR5, CFI and CFP; complement regulators such as C1QBP, CD-6, CD55 and CD59; or cathepsins such as CTSB, CTSC, CTSD, CSTL, CSTO, or CTSS or any combination thereof) or alternatively, the desired effect on the underlying disease state (for example, an amount sufficient to inhibit tumor growth in a subject) in at least a sub-population of cells in a subject at a reasonable benefit/risk ratio applicable to any medical treatment. Determination of therapeutically effective amounts of the agents used in this invention, can be readily made by one skilled in the art, by the use of known techniques and by observing results obtained under analogous circumstances. The amounts/dosages may be varied depending upon the requirements of the subject in the judgment of the treating clinician; the severity of the condition being treated and the particular composition being employed. In determining the therapeutically effective amount, a number of factors are considered by the treating clinician, including, but not limited to: the specific disease state; pharmacodynamic characteristics of the particular agent and its mode and route of administration; the desired time course of treatment; the species being treated; its size, age, and general health; the specific disease involved; the degree of or involvement or the severity of the disease; the response of the individual patient; the particular agent administered; the mode of administration; the bioavailability characteristics of the preparation administered; the dose regimen selected; the kind of concurrent treatment (i.e., the interaction of the agent with other co-administered agents); and other relevant circumstances.

For example, as described herein, the amino acid sequence of the C3d protein can be truncated/mutatedlaitered to produce biologically active peptides or variants. Such peptides derived from the C3d protein can be synthesized, or otherwise produced and evaluated for their biological activity. Biological activity can include binding of C3d or C3d peptides to MHC, or change of sites of proteolysis by proteases such as metalloproteinases. Mutations can specifically increase MHC binding to increase immunostimulation. Althemafively, other immunostimulatory peptides can be used.

In certain embodiments, the agents described for use in this invention can be combined with other pharmacologically active compounds (“additional active agents”) known in the art according to the methods and compositions provided herein. Additional active agents can be large molecules (e.g., proteins, lipids, carbohydrates), or other immunostimulatory peptides or small molecules (e.g., synthetic inorganic, organometallic, or organic molecules). In one embodiment, additional active agents independently or synergistically help to treat cancer.

For example, certain additional active agents are anti-cancer chemotherapeutic agents. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxonibicin, and mitomycin; and vinca alkaloid natural antineoplastics, such as vinblastine and vincristine or agents targeted at specific mutations within tumor cells.

Further, the following drugs may also be used in combination with an antineoplastic agent, even if not considered antineoplastic agents themselves: dactinomycin; daunorubicin HCl; docetaxel; doxoruhicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT), For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used in conjunction with the invention: checkpoint inhibitors that target for example, PD-1 and CTLA-4, interleukins 1 through 37, including mutants and analogues; interferons or cytokines, such as interferons .alpha., .beta., and .gamma.; hormones, such as luteinizing hormone releasing hormone (MBE) and analogues and, gonadotropin releasing hormone (GnRH); growth factors, such as transforming growth factor-.beta. (TGF-.beta.), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-.alpha. & .beta. (TNF-.alpha. & .beta.); invasion inhibiting factor-2 (IIF-2); bone morphogenetic proteins 1-7 (BMP 1-7); somatostatin; thymosin-.alpha.-1; .gamma.-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, tneturedopa, and uredopa; ethylenimines and methylamelamines including altretamine, triethylenemelamine, trietylenephosphoramide, triethiylenethiophosphoramide and ttimethylolotnelamine; acetogenins (especially bullata.cin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal 1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bileomycins, cactinomycin, cara.bicin, caminomycin, carzinophilin, chromomycinis, dactinomycin, daunorubicin, detorubicin, 6-diazo-5-oxo-L-norleucine, doxorubicin (including morpholine-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenitnex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2. toxin, verracurin roridin A and amidine); urethan; vindesine; dacarbazine; mannomustine; mitobronitol; mitolactol; pipobroman; gacytosine; arabinoside (“Ara-C”); cyclophosphamide; thiotepa; taxoids, paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexa.te; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

The compositions and methods of the invention can comprise or include the use of other biologically active substances, including therapeutic drugs or pro-drugs, for example, other chemotherapeutic agents or antigens useful for cancer vaccine applications. Various forms of the chemotherapeutic agents and/or additional active agents may be used. These include, without limitation, such forms as uncharged molecules, molecular complexes, salts, ethers, esters, amides, and the like, which are biologically active.

The agents and substances described herein can be delivered to the subject in a pharmaceutically suitable, or acceptable or biologically compatible carrier. The terms “pharmaceutically suitable/acceptable” or “biologically compatible” mean suitable for pharmaceutical use (for example, sufficient safety margin and if appropriate, sufficient efficacy for the stated purpose), particularly as used in the compositions and methods of this invention.

The compositions described herein may be delivered by any suitable route of administration for treating the cancer, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, through an inhalation spray, or other modes of delivery known in the art.

Previously it has been reported that a deficiency of Complement Component C3 in mouse models of ovarian cancer was associated with reduced tumor growth. Deficiencies of receptors for C3a and C5a in the tumor also slowed tumor growth. Analysis of The Cancer Genome Atlas-NIH (TCGA) data confirmed that lowered expression of C3 in human tumors was associated with better survival, supporting the mouse data. Bioinformatic analysis of the mechanism identified C3 as an on/off switch for the immune system where one breakdown product iC3b promoted immunosuppressi on whereas another proteolytic product C3dg/C3d was an immune stimulant. C3dg and C3c1 are proteolytically produced from iC3b and its production is inhibited by regulatory proteins that hide the sites where proteolysis occurs. Without wishing to be bound by theory, it is reasonable to believe that replacing full length C3 produced by the tumor with its C3dg or C3d product or a biologically active peptide derived from C3d or other immunostimulatory peptides would make the tumor immunogenic by removing the iC3b immunosuppressive shield allowing the immune system to see the unique tumor associated antigens while at the same time stimulating an immune response through the actions of C3dg, C3d or a peptide derived from C3d or other immunostimulatory peptides. This combined approach shifts the immune response to a state where the tumor is rejected. It takes the tumor from “cold” mode where there is no immune response to “hot” mode where the immune system attacks the tumor. When in “hot” mode, other immune modulators that are clinically approved can be used to enhance the response.

As described herein, various methods can be used to knock-down (partially inhibit or decrease) or knock-out (completely inhibit or abrogate) the expression, activity or production of the C3 gene, or a fragment of the C3 gene, in a tumor cell, with the result that its biologically active breakdown product, iC3b, cannot provide an immunosuppressive shield for the tumor. A list of genes that are suitable for knock-down, or knock-out targeting in the present invention, and their sequences are as follows.

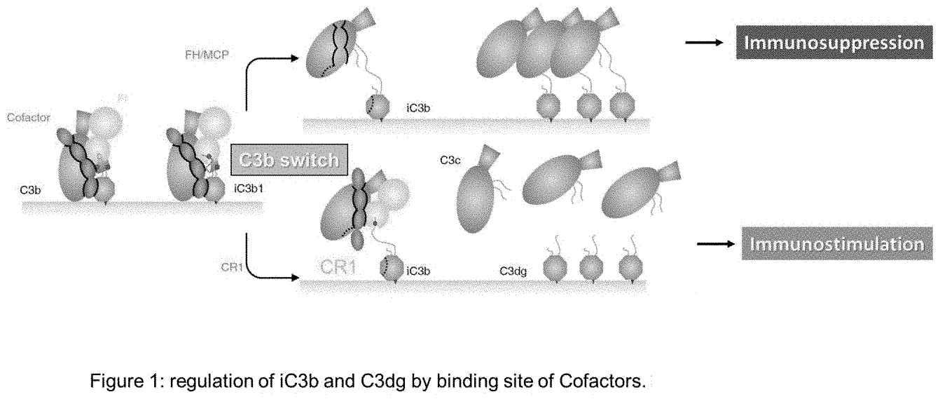

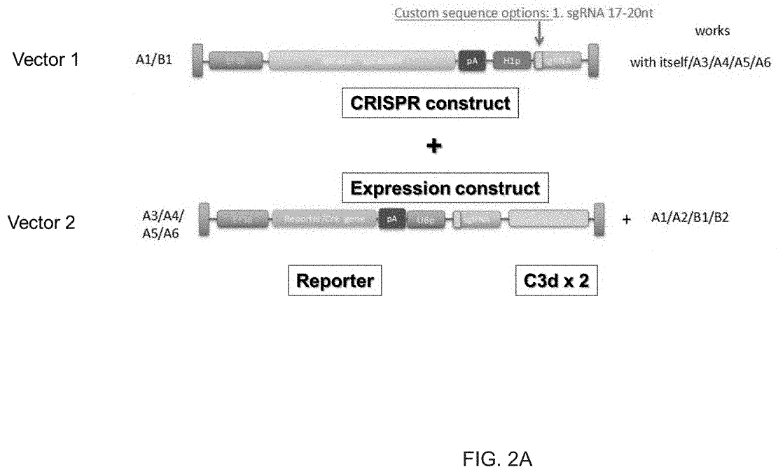

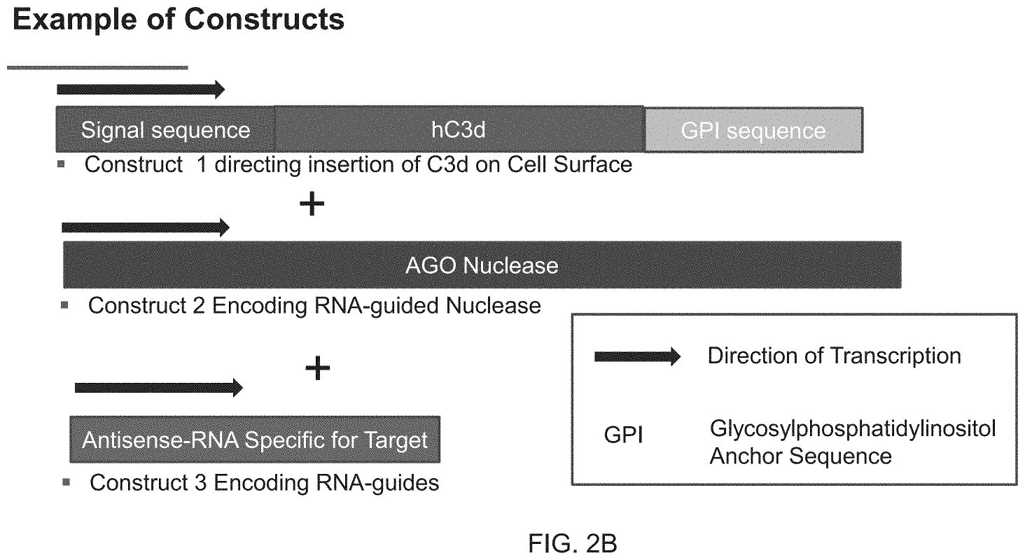

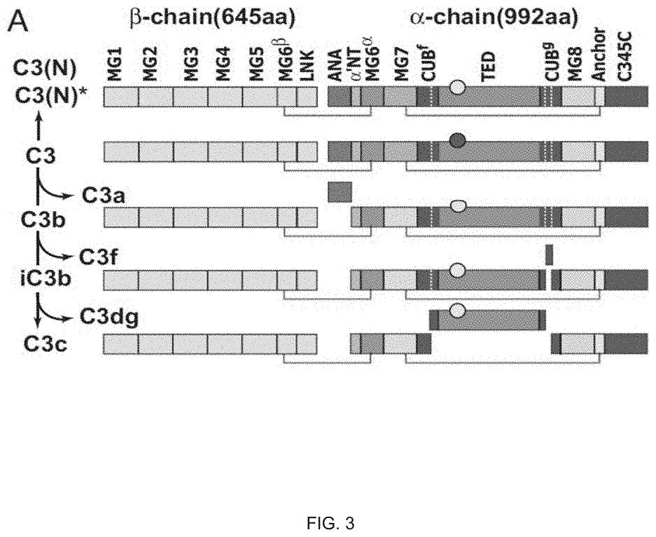

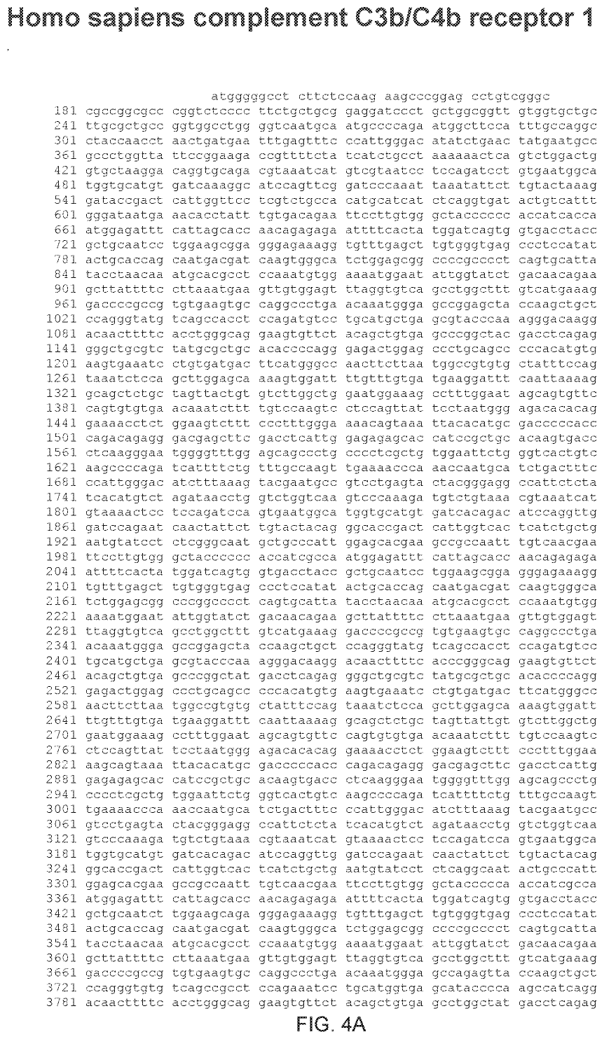

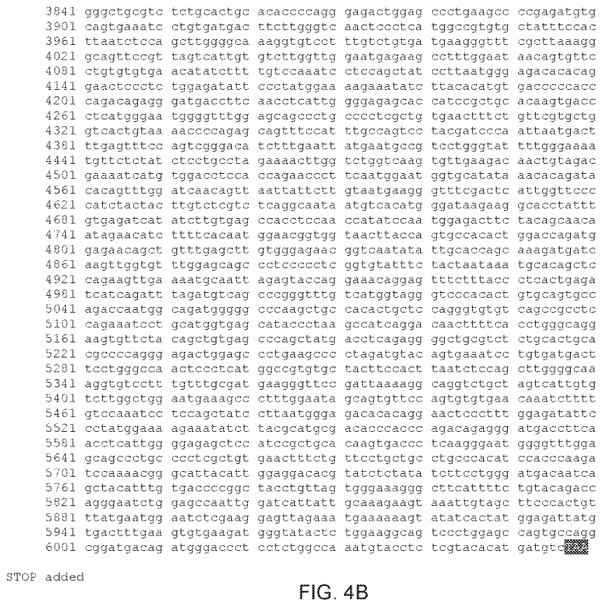

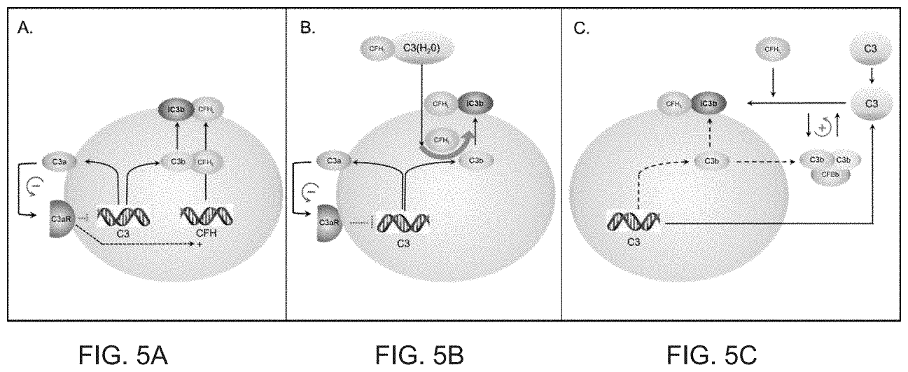

The nucleic acid sequence for C3, including the fragments C3d and C3dg can be found e.g., in Proc. Natl. Acad. Sci. USA, vol. 82, pp. 708-712, February 1985. The term “C3d” as used herein is intended to encompass both C3d and C3dg, The nucleic acid sequence for the C3aR can be found at “C3AR1 complement C3a receptor 1 [Homo sapiens (human)]” Gene ID: 719, ncbi.nlm.nih.gov/gene, updated on 6 Aug. 2017. The nucleic acid sequence for the C5a receptor can be found at “C5AR1 complement C5a receptor 1 [Homo sapiens (human)]” Gene ID: 728, ncbi.nlm.nih.gov/gene, updated on 29 Aug. 2017. C1R complement C1r [Homo sapiens (human)], Gene ID: 715, ncbi.nlm.nih.gov/gene, updated on 3 Sep. 2017, C1RL complement C1r subcomponent like [Homo sapiens (human), Gene ID: 51279, ncbi.nlm.nih.gov/gene, updated on 3 Sep. 2017, C5AR2 complement component 5a receptor 2. [Homo sapiens (human)], Gene ID: 27202. ncbi.nlm.nih.gov/gene, updated on 3 Sep. 2017, C1QBP complement C1q binding protein [Homo sapiens (human)}, Gene ID: 708, ncbi.nlm.nih.gov/gene, updated on 3 Sep. 2017, CR2 complement C3d receptor 2 [Homo sapiens (human)], Gene ID: 1380, ncbi.nlm.nih.gov/gene, updated on 3 Sep. 2017, CD46 molecule [Homo sapiens (human)], Gene ID: 4179, ncbi.nlm.nih.gov/gene, updated on 3 Sep. 2017, CD55 molecule (Cromer blood group) [Homo sapiens (human)], Gene ID: 1604, ncbi.nlm.nih.gov/gene, updated on 6 Sep. 2017, CD59 molecule (CD59 blood group) [Homo sapiens (human)], Gene ID: 966, ncbi.nlm.nih.gov/gene, updated on 3 Sep. 2017 and LAIR1 leukocyte associated immunoglobulin like receptor 1 [Homo sapiens (human)], Gene ID: 3903, ncbi.nlm.nih.gov/gene, updated on 3 Sep. 2017 Complement factor B, CFB [Homo sapiens (human)], Gene ID: 629, ncbi.nlm.nih.gov/gene, updated on 3 Sep. 2017, complement factor D, CFD, [Homo sapiens (human)], ID: 1675, ncbi.nlm.nih.gov/gene, updated on 3 Sep. 2017; complement factor H, CFH, [Homo sapiens (human)]; Gene ID: 3075, ncbi.nlm.nih.gov/gene, updated on 3 Sep. 2017; complement factor H related 1, CFHR1, [Homo sapiens (human)], Gene ID: 3078, ncbi.nlm.nih.gov/gene, updated on 3 Sep. 2017; complement factor H related 2, CFHR2, [Homo sapiens (human)], Gene ID: 3080, ncbi.nlm.nih.gov/gene, updated on 3 Sep. 2017; complement factor H related 3, CFHR3, [Homo sapiens (human)], Gene ID: 10878, ncbi.nlm.nih.gov/gene, updated on 3 Sep. 2017; complement factor H related 4, [Homo sapiens (human)], Gene ID: 10877, ncbi.nlm.nih.gov/gene, updated on 3 Sep. 2017; complement factor H related 4, CFHR5, [Homo sapiens (human)], Gene ID: 81494, ncbi.nlm.nih.gov/gene, updated on 3 Sep. 2017, complement factor I, CFI, [Homo sapiens (human)], Gene ID: 3426, ncbi.nlm.nih.gov/gene, updated on 3 Sep. 2017; complement factor properdin, CFP, [Homo sapiens (human)], Gene ID: 5199, ncbi.nlm.nih.gov/gene, updated on 3 Sep. 2017; Cathepsin B, CTSB, [Homo sapiens (human)], Gene ID: 1508, ncbi.nlm.nih.gov/gene, updated on 3 Sep. 2017; Cathepsin C, CTSC, [Homo sapiens (human)], Gene ID: 1075, ncbi.nlm.nih.gov/gene, updated on 3 Sep. 2017; Cathepsin D, CTSD [Homo sapiens (human)], Gene ID: 1509, ncbi.nlm.nih.gov/gene, updated on 3 Sep. 2017; Cathepsin L, CSTL, [Homo sapiens (human)], Gene ID: 1514, ncbi.nlm.nih.gov/gene, updated on 3 Sep. 2017; Cathepsin O, CSTO, [Homo sapiens (human)], Gene ID: 1519, ncbi.nlm.nih.gov/gene, updated on 3 Sep. 2017; Cathepsin S, CSTS, [Homo sapiens (human)], Gene ID: 1520, ncbi.nlm.nih.gov/gene, updated on 3 Sep. 2017. For example, a gene editing technique to inactivate the C3 gene within tumors can be used (see e.g., U.S. Pat. No. 8,697,359 for a description of CRISPR techniques). Delivery of CRISPR/CAS9 with a sgRNAs to C3 (excluding the C3d sequence) and the nucleic acid sequences for C3d or C3d derived peptides or other immunostimulatory peptides, to a tumor cell can be provided by use of a viral vector. A number of viral vectors have been used in humans and these can be used to transduce the genetic material in different cell types. Such methods are known to those of skill in the art. Means to target the vectors for specific delivery of the constructs to the tumor cells of interest are also known to those of skill. For example, genetically engineered vectors exist where the capsid is modified to contain ligands for receptors that facilitate viral entry onto a particular cell type. An example is given in FIG. 1. This construct also includes a reporter gene that allows efficiency of transduction of the virus into the tumor to be quantitated.

Rather than knocking out C3 (complement components, such as C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, the intracellular expression of these genes can be suppressed in the tumor cell by the expression of a protein that inhibits transcription of the C3 gene. Alternatively, another protein that binds to C3 and leads to its destruction or inhibits processing of C3 can be introduced and expressed in the cell. These include intracellular antibodies, nanobodies or other engineered proteins as well as inhibitors of the cellular proteolytic machinery such as ubiquitin ligases or proteolytic enzymes. A micro-RNA can be expressed that prevents translation of C3. An oligonucleotide composed of a mix of ribonucleotide, deoxyribonucleotide or modified bases that destabilizes C3 RNA or inhibits its translation can be expressed or introduced into the tumor to prevent C3 production, Such methods are known to those of skill in the art.

To stimulate the immune response, C3d can be expressed as the minimal domain, an extended domain, as a monomer or a multimer consisting of repeats of the core C3d sequence with, or without, modifications to C3d amino acids designed to enhance its adjuvant effects. Immunostimulatory peptides (i.e., biologically active peptides) derived from C3d or other immunostimulatory peptides can be expressed as the minimal domain, an extended domain, as a monomer or a multimer consisting of repeats of the core peptide sequence with, or without, modifications to peptide amino acids designed to enhance its adjuvant effects, improve stability or improve pharmacological properties such as half-life within the tumor. Modifications to C3d, or biologically active peptides derived therefrom, would include fusion with other sequences that direct it to particular cellular or extra-cellular locations or to particular binding partners or that also act to stimulate the immune response. Modifications to the thio-ester bond forming residues can be used to render C3d soluble rather than membrane bound. C3d can be also added as a peptide or peptide fusion containing the modifications already listed.

The above approaches can be combined with other cancer therapies including immune-modulators such as checkpoint inhibitor ligands for PD-1 CTLA-4, ICOS, OX40; reagents against C3a and C5a receptors; lymphokines, cytokines and their receptors and strategies designed to increase major and minor histocompatibility antigens. Additionally, the methods of the present invention can be combined with other standard cancer therapies such as radiotherapy and chemotherapy.

### EXAMPLES

### Example 1: Method to Select Immune-Active Peptides from C3d

The method described herein and in Example 2 is based upon the approach of Knopf, P. M. et al,, Immunol. Cell Biol. (2008) 86, 221-225, and De Groot, A. S., Immunol. Cell Biol. (2014) 1-9 to identify immunostimulatory Class II MHC binding petides present in C3d, This approach can be extended to identity other peptides suitable for use in the present invention. The aims of these extensions are:
1. to identify high affinity Class I and Class II Major Histocompatibility Complex (MHC) binding sites for C3d derived peptides using non-proprietary algorithms
2. to ensure that metalloprotease in the tumor environment can digest them to the correct size for binding to MHC proteins

### Procedure

1. The human C3d amino acid sequence: (SEQ ID NO: 5)

HLIVTPSGCGEQNMIGMTPTVIAVHYLDETEQWEKFGLEKRQGALELIKK
GYTQQLAFRQPSSAFAAFVKRAPSTWLTAYVVKVFSLAVNLIAIDSQVLC
GAVKWLILEKQKPDGVFQEDAPVIHQEMIGGLRNNNEKDMALTAFVLISL
QEAKDICEEQVNSLPGSITKAGDFLEANYMNLQRSYTVAIAGYALAQMGR
LKGPLLNKFLTTAKDKNRWEDPGKQLYNVEATSYALLALLQLKDFDFVPP
VVFRWLNEQRYYGGGYGSTQATFMVFQALAQYQKDAPDHQELNLDVSLQL
PSR

was aligned with sequences from dog, pig, cow, mouse and rat to identify conserved regions.
2. The human sequence was also entered into the webservers at:

a. tools.immuneepitope.org/mhci/

b. tools.immuneepitope.org/mhcii/

and screened for predicted binding to the following Class I and Class II MHC alleles:

| Class I alleles | Class II alleles |
|---|---|
| HLA-A*01:01 | HLA-DRB1*01:01 |
| HLA-A*01:01 | HLA-DRB1*03:01 |
| HLA-A*02:01 | HLA-DRB1*04:01 |
| HLA-A*02:01 | HLA-DRB1*04:05 |
| HLA-A*02:03 | HLA-DRB1*07:01 |
| HLA-A*02:03 | HLA-DRB1*08:02 |
| HLA-A*02:06 | HLA-DRB1*09:01 |
| HLA-A*02:06 | HLA-DRB1*11:01 |
| HLA-A*03:01 | HLA-DRB1*12:01 |
| HLA-A*03:01 | HLA-DRB1*13:02 |
| HLA-A*11:01 | HLA-DRB1*15:01 |
| HLA-A*11:01 | HLA-DRB3*01:01 |
| HLA-A*23:01 | HLA-DRB3*02:02 |
| HLA-A*23:01 | HLA-DRB4*01:01 |
| HLA-A*24:02 | HLA-DRB5*01:01 |
| HLA-A*24:02 | HLA-DQA1*05:01/DQB1*02:01 |
| HLA-A*26:01 | HLA-DQA1*05:01/DQB1*03:01 |
| HLA-A*26:01 | HLA-DQA1*03:01/DQB1*03:02 |
| HLA-A*30:01 | HLA-DQA1*04:01/DQB1*04:02 |
| HLA-A*30:01 | HLA-DQA1*01:01/DQB1*05:01 |
| HLA-A*30:02 | HLA-DQA1*01:02/DQB1*06:02 |
| HLA-A*30:02 | HLA-DPA1*02:01/DPB1*01:01 |
| HLA-A*31:01 | HLA-DPA1*01:03/DPB1*02:01 |
| HLA-A*31:01 | HLA-DPA1*01/DPB1*04:01 |
| HLA-A*32:01 | HLA-DPA1*03:01/DPB1*04:02 |
| HLA-A*32:01 | HLA-DPA1*02:01/DPB1*05:01 |
| HLA-A*33:01 | HLA-DPA1*02:01/DPB1*14:01 |
| HLA-A*33:01 | |
| HLA-A*68:01 | |
| HLA-A*68:01 | |
| HLA-A*68:02 | |
| HLA-A*68:02 | |
| HLA-B*07:02 | |
| HLA-B*07:02 | |
| HLA-B*08:01 | |
| HLA-B*08:01 | |
| HLA-B*15:01 | |
| HLA-B*15:01 | |
| HLA-B*35:01 | |
| HLA-B*35:01 | |
| HLA-B*40:01 | |
| HLA-B*40:01 | |
| HLA-B*44:02 | |
| HLA-B*44:02 | |
| HLA-B*44.03 | |
| HLA-B*44:03 | |
| HLA-B*51:01 | |
| HLA-B*51:01 | |
| HLA-B*53:01 | |
| HLA-B*53:01 | |
| HLA-B*57:01 | |
| HLA-B*57:01 | |
| HLA-B*58:01 | |
| HLA-B*58:01 | |

1. The percentile rank for each C3d sequence was summed across all Class I alleles and also across all Class II alleles to identify promiscuously binding peptides to increase the probability that they are active in a wide range of individuals.
2. The low scoring pan-MHC C3d sequences were then further screened to identify those that are conserved across species.
3. The conserved pan-MHC sequences were selected for further analysis were then screened for sites subject to proteolysis by metalloproteases (MMP) 2, 14, 15, 16, 24 and 25 at the following website:

protease.burnham.org/www/tools/cgi-bin/specdb/
4. Where necessary, conservative mutations were introduced into the conserved pan-MHC sequences to produce high efficiency proteolysis sites at the end of each MHC binding sequence or to remove proteolysis sites that exited within the sequence. Each mutation was screened at:

protease.burnham.org/lwww/tools/cgi-bin/specdb/
5. This approach enabled multiple Class I and Class II MHC sequences to be combined in a single peptide.
6. Mutated sequences were rescreened for MHC binding to ensure that affinity was preserved.
7. Mutated sequences were screened against the non-redundant human protein database to ensure that they did not align with proteins other than C3, reducing the risk of inadvertently inducing an auto-immune response against these other proteins:

blast.ncbi.nlm.nih.gov/

### Example 2: C3 MHC Peptides

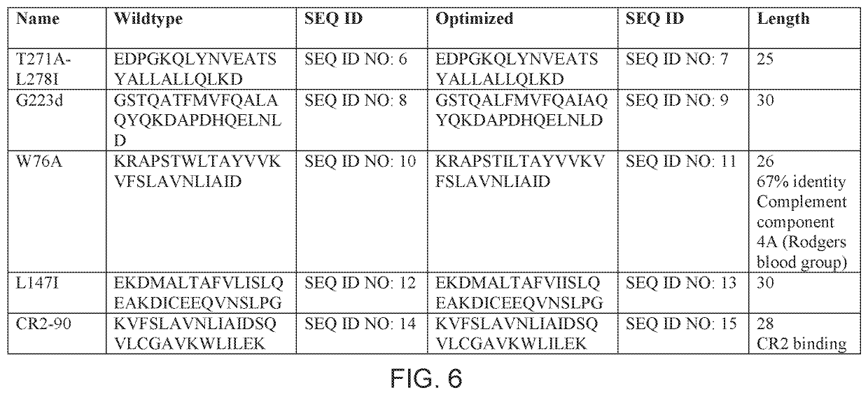

FIG. 6 lists C3 wild type amino acid sequences (SEQ ID NOS: 6, 8, 10, 12 and 14) and their related mutated sequences (SEQ ID NOS: 7, 9, 11, 13 and 15) that have been optimized to improve MHC binding and change sites of proteolysis by metalloproteinases. The mutated peptides reasonably increase MHC binding and increase immune stimulation.

### Example 3: Design of Guide RNA Sequences for CRISPR

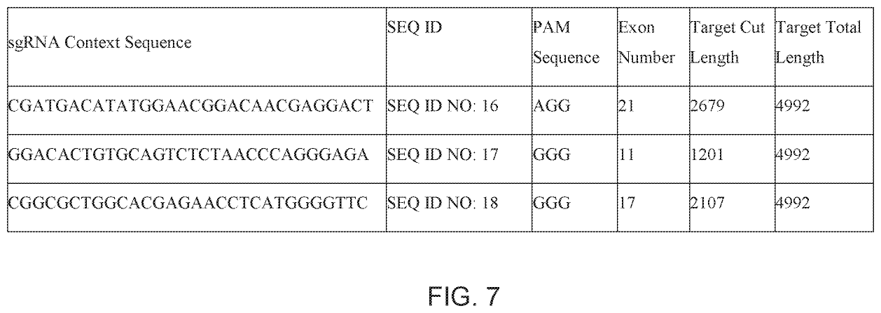

For methods of gene editing using the CRISPR technique, guide RNA sequences are required that reasonably edit/cut out the C3 gene but do not edit/cut out the C3d gene sequence. See for example, portals.broadinstitute.org/gpp/public/analysis-tools/sgrna-design. Exemplary guide RNA sequences are shown in FIG. 7 (SEQ ID NOS: 16-18) along with corresponding PAM sequences.

### Example 4: Anti-Sense RNAs

Gene editing methods as described herein can also be accomplished using interfering RNA sequences. Such sequences can be, for example:

(SEQ ID NO: 19)
5′ ACAUUCUGAUUCCUUCCGG 3′
(SEQ ID NO: 20)
5′ ACUUUGUGCACUCCUUCAC 3′

The RNAs can be synthetized from a single DNA insert withRNAIIl promoters (e.g. U6, 7SL) on either side of the insert. The DNA insert has as its top strand the following sequence with anti sense strands underlined:

(SEQ ID NO: 21)
5′ACATTCTGATTCCCTTCCGGAAGCCGGAAGGAATCAGAATGACATTTT
TTGAGCTCAAAAAATGTCTTTCTGCACTCCTTCACCTTGTGAAGGAGTGC
AGAAAGT 3′.

### Example 5: C3d/CD55 or CD59 Fusion Construct

C3d is produced normally by the proteolytic cleavage of Complement Protein C3. During this process, C3d becomes anchored to the cell membrane through a thioester bond formed mostly with the hydroxyl groups of cell-surface carbohydrates (Law, S. K. and A. W. Dodds, “The internal thioester and the covalent binding properties of the complement proteins C3 and C4.” Protein Science: a publication of the Protein Society 6 (2): 263-274 (1997). C3d is bound by Complement Receptor 2 (CR2) and stimulates the adaptive immune response (Ricklin et al. “The renaissance of complement therapeutics.” Nature Reviews. Nephrology 14 (1): 26-47 (2018).

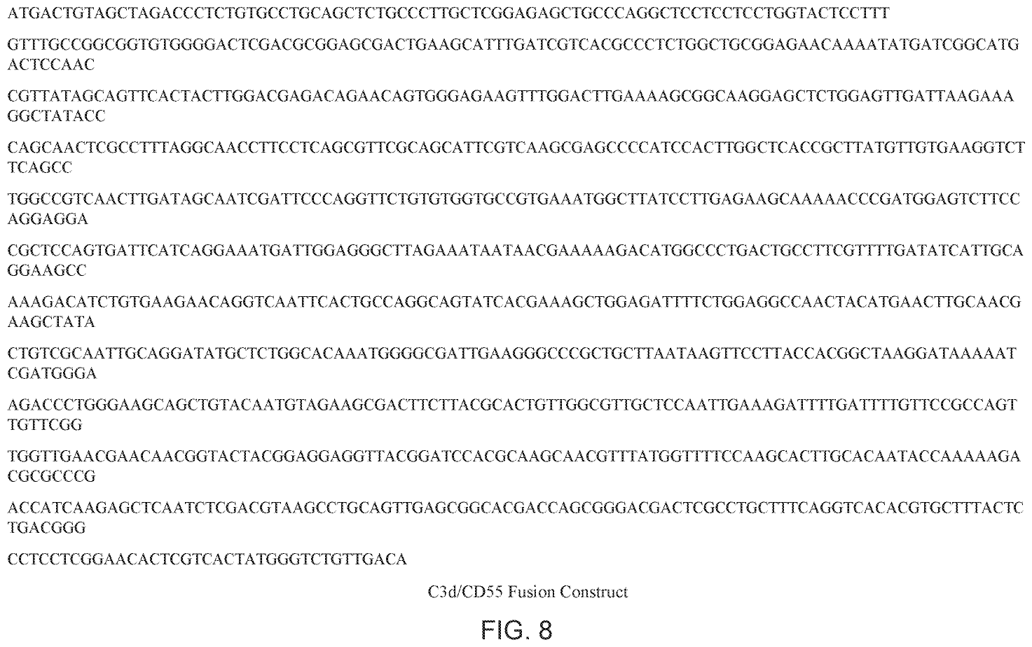

Described herein are methods to increase immunogenicity of antigens using a C3d fusion protein bound to the cell membrane by a Glycosylphosphatidylinositol (GPI) anchor. In this example, a Cd3/CD55 fusion construct (See FIG. 8, SEQ ID NO: 22) has been prepared that can express Cd3 on a cell membrane. Alternatively, a Cd3/CD59 fusion construct can be prepared in a similar manner.

The fusion has three parts:

(1) A signal sequence directing export to the cell surface membrane

(2) The C3d sequence

(3) A sequence directing the attachment of the GPI tag

Over 150 proteins are naturally processed to add a GPI anchor and can be used as described herein as sources for parts 1 and 3 of the fusion protein (Kinoshita, T. and M. Fujita “Biosynthesis of GPI-anchored proteins: special emphasis on GPI lipid remodeling.” Journal of Lipid Research 57 (1): 6-24 (2016).

The present invention uses sequences for Part 1 and 3 from CD55, while removing all other sequence information present in CD55 essential to its function as a regulator of complement activation (Coyne, Crisci et al. “Construction of synthetic signals for glycosyl-phosphatidylinositol anchor attachment. Analysis of amino acid sequence requirements for anchoring.” The Journal of biological chemistry 268 (9): 6689-6693 (1993)). The fusion contains the following sequences, given as single letter amino acid codes, for each part:

(SEQ ID NO: 23)
MTVARPSVPA ALPLLGELPR LLLLVLLCLP AVWG (signal
sequence)
(SEQ ID NO: 24)
LDAERLKHLIVTPSGCGEQNMIGMTPTVIAVHYLDETEQWEKFGLEKRQG
ALELIKKGYTQQLAFRQPSSAFAAFVKRAPSTWLTAYVVKVFSLAVNLIA
IDSQVLCGAVKWLILEKQKPDGVFQEDAPVIHQEMIGGLRNNNEKDMALT
AFVLISLQEAKDICEEQVNSLPGSITKAGDFLEANYMNLQRSYVTVAIAG
YALAQMGRLKGPLLNKFLTTAKDKNRWEDPGKQLYNVEATSYALLALLQL
KDFDFVPPVVRWLNEQRYYGGGYGSTQATFMVFQALAQYQKDAPDHQELN
LDVSLQL (C3d Sequence)
(SEQ ID NO: 25)
SG TTSGTTRLLS GHTCFTLTGL LGTLVTMGLL T (GPI anchor)

Construction of a fusion protein using Part 1 and 3 from CD59 is also possible, as follows:

(SEQ ID NO: 26)
MGIQGGSVLF GLLLVLAVFC HSGHS (signal sequence)
(SEQ ID NO: 24)
LDAERLKHLIVTPSGCGEQNMIGMTPTVIAVHYLDETEQWEKFGLEKRQG
ALELIKKGYTQQLAFRQPSSAFAAFVKRAPSTWLTAYVVKVFSLAVNLIA
IDSQVLCGAVKWLILEKQKPDGVFQEDAPVIHQEMIGGLRNNNEKDMALT
AFVLISLQEAKDICEEQVNSLPGSITKAGDFLEANYMNLQRSYVTVAIAG
YALAQMGRLKGPLLNKFLTTAKDKNRWEDPGKQLYNVEATSYALLALLQL
KDFDFVPPVVRWLNEQRYYGGGYGSTQATFMVFQALAQYQKDAPDHQELN
LDVSLQL (C3d Sequence)
(SEQ ID NO: 27)
ENGGTSLSEK TVLLLVTPFL AAAWSLHP (GPI anchor)

An alternative is to replace part 3 with the processed GPI anchor sequences as described by Nagarathinam, A., P. Hoflinger, et al. “Membrane-anchored Abeta accelerates amyloid formation and exacerbates amyloid-associated toxicity in mice,” The Journal of neuroscience: the official journal of the Society for Neuroscience 33 (49): 19284-19294 (2013). In this case, the sequence for part 3 is:

(SEQ ID NO: 28)
SRDGRRS

References (the teachings of which are incorporated herein by reference:
1. Neoplasia. 2012 November; 14 (11): 994-1004. DOI 10.1593/neo.121262 “Genetic and Pharmacologic Inhibition of Complement Impairs Endothelial Cell Function and Ablates Ovarian Cancer Neovascularization” Nunez-Cruz S, Gimotty P A, Guerra M W, Connolly D C, Wu Y Q, DeAngelis R A, Lambris J D, Coukos G, Scholler N.
2. Cell Rep. 2014 Mar. 27 (6).1085-95, doi: 10,1016/j.celrep.2014.02.014 “Autocrine effects of tumor-derived complement.” Cho M S, Vasquez H G, Rupaimoole R, Pradeep S, Wu S, Land B, Han HD, Rodriguez-Aguayo C, Bottsford-Miller J, Huang J, Miyake T, Choi H J, Dalton H J, Ivan C, Baggerly K, Lopez-Berestein G, Sood A K, Afshar-Kharghan V.
3. JCI Insight. 2017; 2 (9):e90201. doi:10.1172/jci.insight.90201 “C3d regulates immune checkpoint blockade and enhances antitumor immunity” Platt J L, Silva I, Balin S J, Lefferts A R, Farkash E, Ross T M, Carroll M C, Cascalho M.
4. Nat Strut Mol Biol. 2017 Jul. 3. doi: 10.1038/nsmb.3427 Regulator-dependent mechanisms of C3b processing by factor I allow differentiation of immune responses. Xue X, Wu J, Ricklin D, Forneris F, Di Crescenzio P, Schmidt C Q, Granneman J, Sharp T H, Lambris I D, Gros P.
5. Cascalho and Platt: eurekalert.org/pub_releases/2017-05/mmu-fcr051117.php
6. Vaccine compositions and methods of modulating immune responses US 20030133942.
7. Opsonin-enhanced cells, and methods of modulating an immune response to an antigen WO 1997035619 A1.
8. Mutable vaccines U.S. Pat. No. 7,776,321 B2.
9. Dempsrey et al. (1996) Nature 271:348-350.
10. Compositions and methods for generating an immune response US 20150231227 A1.
11. Antibody targeting cell surface deposited complement protein C3d and use thereof WO2015105973 A1.
12. Antibodies to the C3d fragment of complement component 3 US 20130129728 A1.
13. Modulating the immune response CA 2207000 A1.
14. Gene editing for immunological destruction of neoplasia US 20170020922 A1.
15. Methods and compositions for selectively eliminating cells of interest WO 2.016094888 A1.
16. Crispr/cas-mediated genome editing to treat egfr-mutant lung cancer US 20170145405 A1.
17. Cancer therapy U.S. Pat. No. 9,603,912 B2.
18. Prmt5 inhibitors for the treatment of cancer with reduced mtap activity WO 2016145150 A3.
19. Fritsche, L. G., N. Lauer, et al. (2010). “An imbalance of human complement regulatory proteins CFHR1, CFHR3 and factor H influences risk for age-related macular degeneration (AMD).” Human molecular genetics 19 (23): 4694-4704.
20. JnBaptiste, C. K., A. M. Gurtan, et al. (2017). “Dicer loss and recovery induce an oncogenic switch driven by transcriptional activation of the oncofetal Imp1-3 family.” Genes & development 31 (7): 674-687.
21. Liszewski, M. K., M. Kolev, et al. (2013). “Intracellular complement activation sustains T cell homeostasis and mediates effector differentiation.” Immunity 39 (6): 1143-1157.
22. Martin, M., J. Leffler, et al. (2016). “Factor H uptake regulates intracellular C3 activation during apoptosis and decreases the inflammatory potential of nucleosomes,” Cell death and differentiation 23 (5): 903-911.
23. Elvington, M., M. K. Liszewski, et al. (2017) “A C3(H20) recycling pathway is a component of the intracellular complement system.” The Journal of Clinical Investigation 127 (3): 970-981.

While this invention has been particularly shown and described with references to preferred embodiments thereof, it will be understood by those skilled in the art that various changes in form and details may be made therein without departing from the scope of the invention encompassed by the appended claims.

1. A method of enhancing the immunogenicity of a tumor cell, wherein the tumor cell expresses complement protein C3 and/or C5, or complement protein receptor C3aR and/or C5aR, or any combination of said proteins or receptors thereof, the method comprising:
a) contacting the tumor cell with a first agent wherein the first agent decreases the expression, activity or production of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, in the tumor cell, and
b) contacting the tumor cell with a second agent wherein the second agent increases the expression or activity in the tumor cells or the tumor cell microenvironment of complement protein C3d or a biologically active variant thereof including peptides derived from C3d, or other immunostimulatory peptides.

2. The method of claim 1, wherein the first agent comprises a gene-editing agent that decreases or inhibits the expression, activity or production of one, or more complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell.

3. The method of claim 2, wherein the gene-editing agent comprises a CRISPR-Cas system construct that decreases or inhibits the expression, activity or production of one, or more, complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell.

4. The method of claim 2, wherein the gene-editing agent comprises a TALEN construct that decreases or inhibits the expression, activity or production of one, or more complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP;
complement regulators such as C1QBP, CD46, CD55 and CD59; or cathepsins such as CTSB, CTSC, CTSD, CSTL, CSTO, or CTSS or any combination thereof in the tumor cell.

5. The method of claim 3 wherein the gene-editing agent does not decrease or inhibit the expression of C3d or peptides derived from C3d in the tumor cell or other immunostimulatory peptides.

6. The method of claim 1, wherein the first agent is a nucleic acid construct comprising RNAi, shRNA, miRNA or anti-sense RNA that decreases or inhibits the expression, activity or production of one, or more complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell.

7. The method of claim 1, wherein the first agent is a nucleic acid construct that expresses a protein that decreases or inhibits the transcription of one, or more complement components, such as C3 and C5; complement receptors such as C3aR1, C5aR1, C5aR2, C1R, C1RL, CR2 and LAIR1; complement factors such as OFB, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof in the tumor cell.

8. The method of claim 2, wherein the agent is targeted for delivery to the tumor cell using a viral vector, nanoparticle, liposome or exosotne.

9. The method of claim 8 wherein the viral vector comprises adenovirus, adeno-associated virus, a lentiviral vector, a vaccinia virus, a herpes virus vector, a paromxyovirusor or any viral vector or any virus-like particle.

10. The method of claim 1, wherein the second agent comprises an expression vector that targets the tumor cell, wherein the vector comprises a nucleic acid construct that expresses C3d, or a biologically active variant thereof including peptides derived from C3d, or encodes a protein that activates the expression of C3d in the tumor cell or other immunostimulatory peptides.

11. A method of inhibiting tumor growth in a subject, wherein the tumor comprises tumor cells that express complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, or any combination of said proteins or receptors thereof, the method comprising:
a) administering to the subject a therapeutically effective amount of a first agent wherein the first agent decreases the expression of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR4, CFHR5, CFI and CFP; complement regulators such as C1QBP, CD46, CD55 and CD59; or cathepsins such as CTSB, CTSC, CTSD, CSTL, CSTO, or CTSS, or any combination thereof, in the tumor cells, and
b) administering to the subject a therapeutically effective amount of a second agent wherein the second agent increases the expression, activity or production of complement protein C3d or a biologically active variant thereof including peptides derived from C3d, or other immunostimulatory peptides, in the tumor cells or the tumor micro-environment, thereby inhibiting the tumor growth in the subject.

12. The method of claim 11, wherein the subject is a mammal.

13. The method of claim 12, wherein the mammal is a human.

14-25. (canceled)

26. A method of treating cancer, or preventing metastasis of cancer, in a subject, wherein the cancer cells express complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, the method comprising:
a) administering to the subject a therapeutically effective amount of a first agent wherein the first agent decreases the expression, activity or production of complement components, such as C3 and C5; complement receptors such as C3aR1, C5aR1, C5aR2, C1R, C1RL, CR2 and LAIR1; complement factors such as CFB, CFD, CFH, CFHR1, CFHR2, CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, in the cancer cells, and
b) administering to the subject a therapeutically effective amount of a second agent wherein the second agent increases the expression of complement protein C3d or peptides derived from within C3d or other immunostimulatory peptides in the cancer cells or tumor micro-environment, thereby treating cancer, or preventing metastasis of cancer, in the subject.

27-38. (canceled)

9. The method of claim 26, wherein the first and/or second agent is administered concurrently with, or sequentially before or after at least one other cancer treatment.

40. The method of claim 39, wherein the cancer treatment is administration of a treatment selected from the group consisting of: a checkpoint inhibitor; a proteasome inhibitor; immunotherapy; radiation therapy; chemotherapy.

41. A pharmaceutical composition comprising a therapeutically effective amount of a first agent that decreases the expression, production or activity of complement components, such as C3 and C5; complement receptors such as C3aR1, C5aR1, C5aR2, C1R, C1RL, CR2 and LAIR1; complement factors such as CFB, CFD, CFH, CFHR1, CFHR2 CFHR3, CFHR4, CFHR5, CFI and CFP; complement regulators such as C1QBP, CD46, CD55 and CD59; or cathepsins such as CTSB, CTSC, CTSD, CSTL, CSTO, or CTSS or any combination thereof, in a tumor cell, and a therapeutically effective amount of a second agent that increases the expression or activity of complement protein C3d or a biologically active variant thereof including peptides derived from C3d or other immunostimulatory peptides in the tumor cell or the tumor cell microenvirontnent, in a pharmaceutically acceptable medium.

42-53. (canceled)

54. The method of claim 1, wherein the second agent is a fusion protein construct comprising C3d and CD 55, or C3d and CD 59 proteins.

55. The composition of claim 41, wherein the second agent is a fusion protein construct comprising C3d and CD 55, or C3d and CD 59 proteins.

56. The method of claim 4 wherein the gene-editing agent does not decrease or inhibit the expression of C3d or peptides derived from C3d in the tumor cell or other immunostimulatory peptides.
